(19) United States
(12) Patent Application Publication
(10) Pub. No.: US 2011/0189298 A1
(43) Pub. Date: Aug. 4, 2011

(54) DOUBLE EMULSION AND METHOD TO PRODUCE SUCH

(76) Inventors: Hendrik Vos, Rouveen (NL); Albert Thijs Poortinga, Apeldoorn (NL)

(21) Appl. No.: 13/122,084

(22) PCT Filed: Oct. 1, 2009
(86) PCT No.: PCT/NL2009/050593

§ 371 Date: Apr. 19, 2011

(30) Foreign Application Priority Data
Oct. 1, 2008 (NL) 2002046

(51) Int. Cl. A61K 47/08; B01J 13/02; A61K 9/14; A61K 9/107

(52) U.S. Cl. 424/489; 264/4.1; 514/772

(57) ABSTRACT

The invention is directed to a method to produce a water-in-oil-in-water (w/o/w) emulsion, comprising: a. preparing a water-in-oil (w/o) emulsion; b. atomizing said w/o emulsion in the presence of a carrier material comprising at least a water soluble matrix material and at least one emulsifier, to form agglomerates; c. dispersing said agglomerates in an aqueous liquid, such as water or an aqueous solution. Also provided is a new instant powder (obtained after step b) that can be used to prepare the w/o/w emulsion. The emulsion of the invention is advantageously suited for the encapsulation of active components.

### FIELD OF THE INVENTION

The invention relates to the field of preparing emulsions, more specifically for the encapsulation of components. In particular the invention relates to making double emulsions of the water-oil-water (w/o/w) type.

### BACKGROUND

In many applications in the food and pharmaceutical field there is a need to encapsulate water soluble components. The reason for this need is, for instance, to mask the taste of the water soluble component or to prevent chemical reactions between the water soluble components and further ingredients.

Double emulsions of the water-in-oil-in-water type (w/o/w emulsions) would be a solution for this problem. A w/o/w emulsion consists of droplets of oil in an aqueous environment in which the oil droplets are filled with water droplets in which hydrophilic components can be solved. Many examples of w/o/w emulsions are known in the literature (see e.g. Garti, N. and Lutz, R., 2004, Interface Sci. Technol. 4:557-605). In general, a double emulsion can be made by first dispersing water in oil, thereby creating a water-in-oil emulsion and subsequently dispersing this emulsion in water (Matsumoto et al., 1976, J. Colloid Interference Sci. 57:353-361). A common problem in preparing these type of emulsions is found in the behavior of the inner water droplets which tend to ‘escape’ to the outer water phase because of thermodynamic instability. This behavior not only occurs at the moment of preparing the emulsion but also during storage. Lifetime of double emulsions therefore is rather low. This could be solved by drying the double emulsion by, for instance, spray drying. However, the forces that are involved with spray drying would cause the emulsion to disintegrate. Therefore, there is still need for a process to obtain a double emulsion that can be stored in powder form.

### SUMMARY OF THE INVENTION

The invention concerns a method to produce a water-in-oil-in-water (w/o/w) emulsion comprising:
a. preparing a water-in-oil (w/o) emulsion;
b. atomizing said w/o emulsion in the presence of a carrier material comprising at least a water soluble matrix material and at least one emulsifier, thereby forming agglomerates;
c. dispersing said agglomerates in an aqueous liquid, such as water or an aqueous solution.

The invention further provides a method to produce a powder comprising
a. preparing a water-in-oil (w/o) emulsion; and
b. atomizing said w/o emulsion in the presence of a carrier material comprising at least a water soluble matrix material and at least one emulsifier, thereby forming agglomerates.

A further part of the invention is a method to encapsulate an active ingredient comprising:
a. preparing a water-in-oil (w/o) emulsion in which said active ingredient is present in the oil or the water phase;
b. spraying said w/o emulsion in the presence of a carrier material comprising at least a water soluble matrix material and at least one emulsifier, to form agglomerates; and
c. optionally dispersing said agglomerates in an aqueous liquid, such as water or an aqueous solution.

The emulsion prepared in step a. of any of said methods generally is an emulsion of only two liquid phases (i.e. a ‘single’ w/o emulsion of an aqueous phase in an oil phase, rather than a double emulsion that is formed of a water phase emulsified in an oil phase, which w/o emulsion is in turn emulsified in another aqueous phase. The w/o/w emulsion (a ‘multiple’ emulsion) is in general only formed when dispersing the agglomerates in a suitable liquid, such as the aqueous liquid. The emulsion prepared in step a. may comprise a solid phase (solid particles). The emulsion prepared in step a. may comprise a gas phase. The solid phase respectively the gas phase may be present in the dispersed phase, the continuous phase or both phases of the emulsion prepared in step a.

Preferably, the water soluble matrix in a method of the invention is chosen from the group comprising lactose, dextrins such as maltodextrins, poly-ethylene glycols, preferably low molecular weight poly-ethylene glycols, silicon dioxide, preferably highly disperse silicon dioxide, starch, modified starch and fatty acid salts. Also preferred for the emulsifier are a protein, preferably selected from the group consisting of casein, whey proteins, lectins, polysorbates (such as Tweens), esters of fatty acids, such as citric acid esters, lactic acid esters, and acetic acid esters, sodium and calcium stearoyl lactylates, and sorbitan esters of fatty acids.

The method of the invention can be used to encapsulate an active component. Thereto, the w/o emulsion of step a) may comprise an active component in the aqueous phase, in the oil phase or in both these phases.

Preferably in said method the atomization and agglomeration is performed in a spray drying tower. Also preferred in said method is the addition of gas to the w/o emulsion before atomization.

In another embodiment, the invention is directed to a w/o/w emulsion obtainable by any of the above methods.

In still a further embodiment, the invention comprises a powder that is obtainable after step b) in a method according to the invention. Said powder preferably comprises agglomerates of oily oil-in-water (o/w) particles and powder particles, wherein the powder particles comprise at least a water soluble matrix material and at least one emulsifier.

Also part of the invention is said powder, wherein the oily o/w particles and the powder particles have an average size of about 1 to about 20 micron.

The invention further relates to the use of a method of the invention for the encapsulation of an active ingredient.

### LEGENDS TO THE FIGURES

FIG. 1 is a light microscopic view of the double emulsion of the invention. It shows relatively large oil droplets (diameter about 20 μm) in an aqueous phase filled with smaller water droplets.

FIG. 2 is a confocal scanning laser microscopic image from the powder obtained using the method claimed herein. Visible is an agglomerate of powder particles (black) and oil droplets (grey) filled with water droplets.

FIG. 3 is a light microscopic view of the double emulsion obtained in Example 2.

### DETAILED DESCRIPTION

Formulating active components in food and pharmaceutical applications is needed to give an optimal utilisation of the component. An ‘active component’ as used herein is defined as a water-soluble or oil-soluble compound or composition that needs to be formulated for application in food or pharmaceuticals. Such an active component may be a small molecular pharmaceutical compound, a peptide, a nucleic acid, a carbohydrate, a mineral, enzymes, microorganisms, any chemical compound that can be used as an additive in food, such as a flavour, an aroma, an antioxidant, a colorant, a perfume, a bio-active (meaning a substance inducing a health benefit), and combinations thereof.

Emulsions, in general, are heterogeneous systems of one immiscible liquid dispersed in another in the form of droplets that usually have a diameter of more than 1 micron (μm). The two liquids are chemically unreactive and form systems characterized by a low thermodynamic stability. In most cases one liquid will be water or another aqueous liquid (but in emulsions generally indicated as the water-phase), while the other liquid will be an oil-type liquid, such as an oil or fat or an organic solvent (but in emulsions generally indicated as the oil phase). Simple emulsions are classified according to the nature of their continuous and dispersed phase. It is custom to set forth the droplet (dispersed) phase first followed by the continuous phase separated by a/mark, i.e. either water(droplets)-in-oil(continuous) (w/o) or oil-in-water (o/w) emulsions. Multiple (or double) emulsions are characterized by listing the primary emulsion set first which is dispersed in a continuous phase. For example in a water-in-oil-in-water (w/o/w) multiple emulsion, a w/o emulsion is dispersed in a water continuous phase. During this dispersion step, for thermodynamic reasons, water droplets are likely to escape from the oil phase to the surrounding continuous water phase. Multiple emulsions are more complex systems as the drops of the dispersed phase themselves contain even smaller dispersed droplets that normally consist of a liquid that is miscible, and in most cases, is identical with the continuous phase. They are, therefore, emulsions of emulsions.

Size of droplets in emulsions may be measured by a wide array of particle size analytical techniques, including laser light scattering, mesh screen classification, Coulter counting method, and settling velocity.

Spray drying, as used herein, is a commonly used technique of drying a liquid feed through a hot gas. Typically, this hot gas is air. The liquid feed can be in the form of solutions, colloids, dispersions or emulsions. This process of drying is a one step rapid process and eliminates additional processing. The liquid feed is pumped through an atomizer device that produces fine droplets into the main drying chamber. Atomizers vary with rotary, single fluid, two-fluid, and ultra-sonic designs. These different styles have different advantages and disadvantages depending on the application of the spray drying required. In many instances a spray nozzle is used in place of an atomizer for a different dispersion rate. The hot drying gas can be passed as a co-current or counter-current flow to the atomizer direction. The co-current flow enables the particles to have a lower residence time within the system and the particle separator (typically a cyclone device) operates more efficiently.

Spray drying often is used as an encapsulation technique by the food and pharmaceutical industries. A substance to be encapsulated (the active component or a solution with the active component) and an amphipathic carrier (usually some sort of modified starch) are homogenized as a suspension. The suspension is then fed into a spray drier, usually a tower heated to temperatures well over the boiling point of water. As the slurry enters the tower, it is atomized. The small size of the drops results in a relatively large surface area which dries quickly. As the water dries, the carrier forms a hardened shell around the load. This spray drying technique has been used in U.S. Pat. No. 5,496,574 to encapsulate an oil-in-water emulsion, whereby the emulsion is spray dried onto a soluble beverage powder, whereupon the continuous aqueous phase of the emulsion desiccated to form capsules. Similar techniques have been described to make a water-in-oil powder (see, e.g. Albertini, B. et al., 2008, Eur. J. Pharm. Biopharm., 69:348-357) or a related technique, indicated as Particle Generation for Supersaturated Solutions (PGSS, see e.g. EP-A-1 569522). In this latter case supercritical CO₂ is solved into the emulsion before spray drying, which during spraying exacerbates from the emulsion and helps the atomization. Further, by the adiabatic effect automatically a cooling is caused which makes air cooling redundant. However, these techniques show a number of disadvantages. The particles that are produced in general are not or only marginally dispersable in water, the fat that is used as the oil phase should be solid at storage temperature and if small particles are needed it is very difficult to separate these from the air stream. These disadvantages can be solved by a technique called Concentrated Powder Form (CPF, see e.g. U.S. Pat. No. 6,440,336, WO 2004/018070 and www.natex.at). In this technique a liquid feed like a solution or an emulsion is sprayed using supercritical CO₂ under addition of a powdery material, like starch, silica or cellulose. The result of the spraying in this case is the formation of agglomerates that are able to be dispersed in water. However, in this case, the oil droplets tend to attract to form large oil slicks, which float on the product, i.e. no stable o/w or w/o/w emulsion is formed.

In the present invention a powdery carrier material is used comprising at least a water soluble matrix material and at least one emulsifier, to form agglomerates. Agglomerates, as used in the present invention, are defined as any loosely connected particles of different substances, wherein the connection is formed by physical and not by chemical binding forces, such as adhesion, gravity, stickiness, etc. The powdery carrier material is agglomerated with a w/o emulsion in a spraying step. Upon dissolution of the thus obtained agglomerates, the oil droplets (filled with water containing optionally an active component) are dispersed in the water without coalescing with each other and without forming an oil/fat film on top of the water.

The atomization in step b) to form the agglomerate may be a spray drying step wherein the agglomerate is formed by coating the w/o emulsion with a carrier material comprising a water soluble matrix material and an emulsifier. In such a spray drying step the w/o emulsion may first be dispersed in gas, e.g. in air, after which the droplets are brought in contact with the carrier material, preferably in the form of a powder. Thus, an agglomerate was obtained comprising a powder comprising a w/o emulsion coated with the carrier material. The spray drying step manages to reduce the risk of damaging the w/o emulsion, e.g. the risk of water escaping the water-phase of the w/o emulsion.

The matrix material should be rapidly solvable in an aqueous liquid and would preferably be chosen from the group of lactose, maltodextrin, poly-ethylene glycol, preferably low molecular weight poly-ethylene glycols, silicon dioxide, preferably highly disperse silicon dioxide, starch, modified starch and magnesium stearate. It was found that agglomerates comprising these matrix materials form particularly stable w/o/w emulsions upon reconstitution of the agglomerates in an aqueous liquid, such as water or an aqueous solution. Without wishing to be bound by any theory, it is expected that due to the rapid solubility of the matrix materials of the above-mentioned group of matrix materials in combination with the presence of the emulsifier the coalescence of w/o droplets may at least partially be prevented upon reconstitution of the agglomerates in an aqueous liquid, such as water or an aqueous solution.

As emulsifier all commonly known emulsifiers which are usable in the food and the pharmaceutical industry can be used. The emulsifier is a surface acting agent, preferably selected from the group consisting of Tween, egg yolk, lecithin, proteins such as caseinate or whey protein, carrageenan, alginates, mannitol, glycerol, sorbitol, Arabic gum, guar gum, xanthan gum, tragacanth, methylcellulose, ethylcellulose, hydroxypropylcellulose, ethylmethylcellulose, carboxymethylcellulose, hydroxypropylmethylcellulose, monoacyl and diacyl glycerols.

The function of the emulsifier is to keep the w/o emulsion in its original form, i.e. preventing the oil droplets from coalescing with each other and from forming an oil film during reconstitution of the agglomerates in an aqueous liquid, such as water or an aqueous solution. The emulsifier, also indicated as stabiliser, thus allows a stable w/o/w emulsion to be formed upon dispersion/dissolution in water of the agglomerates. Preferably surface active compounds like proteins (such as whey protein or caseinate) are used as emulsifier. These compounds will effect their stabilising functions by surrounding the oil phase during dispersion/dissolution of the agglomerates in an aqueous liquid, such as water or an aqueous solution.

In this way the invention comprises a method to form a water-in-oil-in-water (w/o/w) emulsion, comprising:
a. preparing a water-in-oil (w/o) emulsion;
b. atomization of said w/o emulsion in the presence of a carrier material comprising at least a water soluble matrix material and at least one further compound, selected from the group consisting of emulsifiers, to form agglomerates; the agglomerates combined forming a free-flowing powder.
c. dispersing said agglomerates in an aqueous liquid, such as water or an aqueous solution.

The preparation of the w/o emulsion with which the process is started, will be done according to the methods and processes known to a person of skill in the art. Preferably the water phase of the w/o emulsion comprises one or more active ingredients that needs to be encapsulated. The water phase may consist of water or any other aqueous liquid, such as water or an aqueous solution that is suitable for the food and pharmaceutical application. Also the oil phase may comprise any oil, fat or organic solvent that is acceptable in food and/or pharmaceuticals. Examples of oils that can be used are vegetable oils such as sunflower oil, soybean oil, corn oil, cottonseed oil, coconut oil, palpom kernel oil, safflower oil, neobee oil, canola oil, peanut oil, sesame oil, linseed (flax) oil, olive oil, Medium Chain Triglyceride (MCT) oils and the like, and animal fats and oils, such as fish oil and fats, milk fats, and the like. Of course, mixtures of any of these are also usable. When an oil is to be used, it is advantageous to use MCT oil in order to prevent the droplets from being oxidised during storage. The oil phase also contains an emulsifier to stabilise the water droplets in the w/o emulsion produced from the oil. Examples include Span's, Admul WOL and monoglycerides.

Atomization of the w/o emulsion can be performed in any way known to the person skilled in the art. To this end, well-known atomization nozzles such as a whirl chamber type pressure nozzle or a 2-fluid nozzle may be used. The carrier material may be introduced around the atomization nozzle for example by carrying the carrier material in an air stream co-flowing with the spray of atomized droplets. It may be advantageous to inject a gas into the w/o emulsion before atomization. This may help to create small droplets of atomized w/o emulsion and may enhance mixing of the atomized w/o emulsion with the carrier material in order to increase agglomeration. Any gas would be suitable for this purpose, but preferred are N₂ and CO₂, wherein the last may be supercritical CO₂.

The above process may well be performed in a spray drying tower in which case the atomized emulsion is sprayed along with an air stream that surpasses the spray nozzle, in which air stream the carrier material as described above is supplied. In this way, agglomerates of the carrier material particles and oil droplets of the emulsion are formed. These agglomerates form a free flowing powder that is easily dispersed in an aqueous liquid, such as water or an aqueous solution, thereby forming the double emulsion. The air stream helps to mix the atomized emulsion with the carrier material, to increase agglomeration, and transports the agglomerates to the exit of the tower to facilitate collection of the powder. It is not necessary to use a stream of hot air, i.e. to perform a drying action. On the other hand, the stream of air may well be hot e.g. to at least partially dry away the water inside the atomized w/o emulsion.

Another embodiment of the invention relates to the w/o/w emulsions that are obtained by the above mentioned method. The emulsions are characterized in that they are more stable than the double emulsions produced thus far. Double emulsions are thermodynamically stable and hence their shelf-life is limited, e.g. because the inner phase leaks out to the outer water phase. This could be solved by removing the outer water phase by drying as to stop processes of leakage until the moment when the double emulsion is reconstituted for use. However, spray-drying is unsuitable for this because of the high shear involved in the atomization step which will generally destroy the emulsion. The invention described here provides a means of producing a double emulsion in which the outer water phase is absent until the moment of reconstitution. Preferably, the oil droplets have a diameter of about 1 to about 25 μm, but if needed, the spray conditions can be adjusted to have very fine oil particles (<1 μm)

Another embodiment of the invention is formed by the powdery agglomerate that is obtained directly by the spraying/agglomerating. This powder is stable during storage and the double emulsion of the invention can instantly be obtained by dispersing it in an aqueous liquid, such as water or an aqueous solution. Thus, it is usable also as an instant power to be commercialized directly (e.g. for applications in soups or for pharmaceutical suspensions that need to be reconstituted just before use, like in vaccines). This powder thus will comprise fat or oil particles agglomerated with particles comprising a water-soluble matrix material and an emulsifier.

In a specific embodiment, the water phase of the w/o emulsion in step a) comprises water and 1-99 wt. %, preferably 50-99 wt. %, water-soluble particles based on the total weight of the water phase. The water-soluble particles are preferably dispersable in oil. The water-phase may further comprise an active ingredient.

The method of the invention and/or the powder of the invention are usable for encapsulating an active component for use in food and pharmacology. It can be applied to all kinds of foods, for example cereals and derived (optionally muesli, cereals for milk), pastry shop, dairy products, nutritional supplements, sugars and derived (optionally chocolates, sweet, nougat, marzipan), sweet dietary (with low level of calories), in regime foods and for diabetics, oils and derived, milk and derived, eggs, vegetables, fruits, tubers and derived, eatable shafts, snacks, appetizers, eatable roots (optionally licorice), bay and wild products, preserves of fruits, meats, sausages, fish, shellfish and crustaceans and their preserves, alcoholic and non-alcoholic drinks, carbonated or still drinks, juices, syrups, nectars, spices, condiments, pre-cooked foods, pre-processed foods (frozen mass of bread), pizzas, honey, soups, bakery products (including bread, cookies, cakes), desserts, cremes, etc.

Especially preferred is the encapsulation of minerals (e.g. in drinks) so that they cannot chemically react with other ingredients, and the encapsulation of peptides to mask their taste.

Also preferred is the encapsulation of probiotics in order to have the oil phase protect them from harsh environments such as acidic media.

Although the main and more useful embodiments of the invention relate to food and pharmaceuticals, the encapsulation process can be employed for other purposes, in particular to encapsulate herbicides, pesticides, germicides, viricides, attractants, repellents, sterilizers, (recombinant) DNA, nucleic acid derivatives, peptides, marker compounds, toilet chemicals, cosmetics, etc.

### Example 1

A w/o emulsion was made from 25% sucrose, 25% water, 1% Admul WOL (Polyglycerol polyricinoleate supplied by Danisco) and 49% sunflower oil. The aqueous emulsion was dispersed finely in the oil phase by homogenization with a high pressure homogenizer (Niro Soavi) at a pressure of 150/50 bar. Thereafter, the emulsion was spray dried through a high pressure nozzle (whirl chamber type) in a filtermat spray dryer. The liquid flow was 20 L/hr, the spraying pressure 100 bar and the temperature of the emulsion 70° C. Before spraying 10 kg CO₂ per hour was injected into the liquid feed. Around the high pressure nozzle an air stream was created in which 40 kg/hr powder was mixed with the emulsion spray. This powder had an average particle size of 10 μm and consisted of 10% caseinate and 90% maltodextrin 33DE. The temperature of the airstream in the tower was about 25° C.

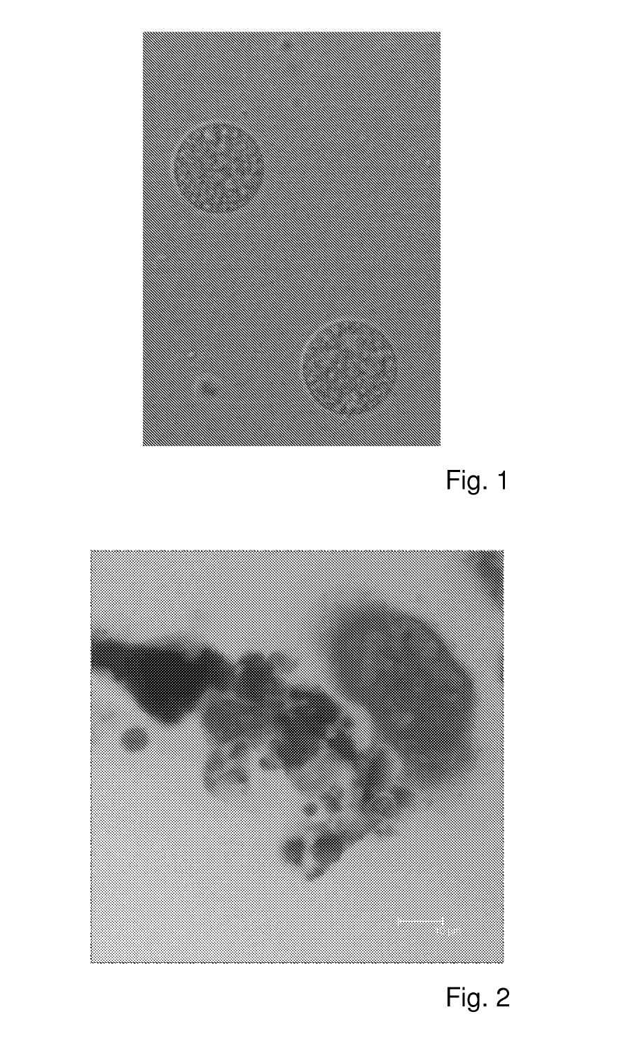

In this way a free flowing powder was created. When 10 grams of this powder were solved into 150 ml water a stable emulsion was formed without any visible oil droplets. Analysis by microscope (FIG. 1) revealed that the emulsion consisted of droplets of about 10 μm filled with small droplets (<1 μm) of water.

Confocal scanning laser microscopy (CSLM) analysis of the powder, in which the oil was stained red, revealed that the powder consisted of an agglomerate of the particles injected through powder injection and oil droplets filled with an aqueous solution (FIG. 2).

### Example 2

A water-in-oil emulsion was prepared with the following composition: 49% sunflower oil, 1% Admul WOL, 24% water, 3% skimmed milk powder, 3% probiotics (BB-12 Bifidobacterium from Chr. Hanssen).

The emulsion was homogenized with a high pressure homogenizer at 150/50 bar and sprayed with a flat body high pressure nozzle (Spraying systems 70/17). Around the nozzle cryogenicall milled Hiprotal 35 (obtained from Friesland Foods Domo) was blown through powder injection, in a ratio of powder: emulsion of 2:1, causing agglomeration with the emulsion and giving a free-flowing powder.

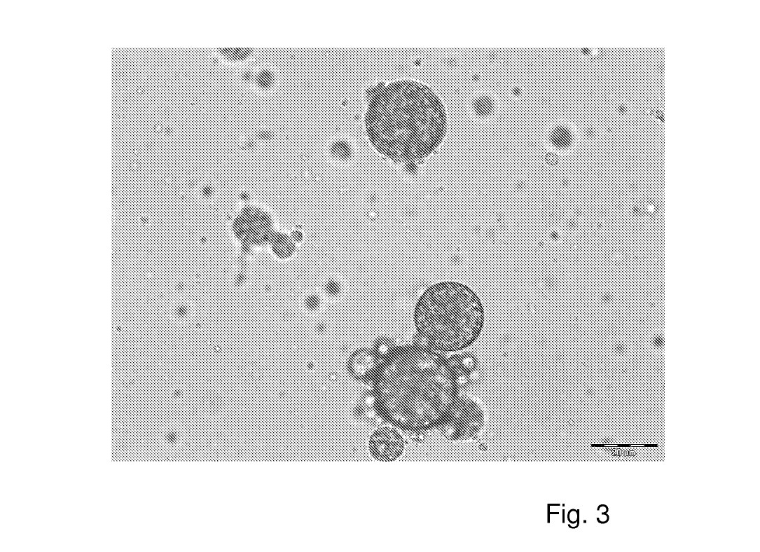

Microscopic analysis of the w/o/w emulsion obtained through dispersing 5% of the powder into water showed that the emulsion comprised oil droplets in which smaller water droplets in which the probiotic was solved (FIG. 3).

To this emulsion HCl was added to pH 2 and the acidified emulsion was kept for 80 minutes at 37° C. to simulate passage through the stomach. The same treatment was applied to a control solution of BB12 powder in water. After incubation, viability of the bacteria was scored: the double emulsion showed 13% survival, while in the control only 1.6% of the bacteria survived.

1. Method to produce a water-in-oil-in-water (w/o/w) emulsion comprising:
a. preparing a water-in-oil (w/o) emulsion;
b. atomizing said w/o emulsion in the presence of a carrier material comprising at least a water soluble matrix material and at least one emulsifier, thereby forming agglomerates;
c. dispersing said agglomerates in an aqueous liquid, such as water or an aqueous solution.

2. Method according to claim 1, wherein the water soluble matrix material is selected from the group consisting of lactose, dextrins such as maltodextrins, poly-ethylene glycols, silicon dioxide, starch, modified starch and fatty acid salts.

3. Method according to claim 1, wherein the emulsifier is selected from the group consisting of proteins, preferably selected from the group consisting of casein, whey proteins and lectins, polysorbates (such as Tweens), esters of fatty acids such as citric, lactic and acetic acid esters, sodium and calcium stearoyl lactylates, and sorbitan esters of fatty acids.

4. Method according to claim 1, wherein the w/o emulsion of step a) comprises an active component in the aqueous phase.

5. Method according to claim 1, wherein the w/o emulsion of step a) comprises an active component in the oil phase.

6. Method according to claim 1, wherein the atomization and agglomeration is performed in a spray drying tower.

7. Method according to claim 6, wherein gas is added to the w/o emulsion before atomization.

8. Method according to claim 1, wherein the water phase of the w/o emulsion in step a) comprises water and 1-99 wt. % water-soluble particles, based on the total weight of the water phase.

9. W/o/w emulsion obtainable by the method according to claim 1.

10. Powder obtainable by step b) in a method according to claim 1.

11. Powder according to claim 10, comprising agglomerates of oily o/w particles and powder particles, wherein the powder particles comprise at least one water soluble matrix material and at least one emulsifier.

12. Powder according to claim 10, wherein the oily o/w particles and the powder particles have an average size of about 1 to about 20 micron.

13. Method to encapsulate an active ingredient comprising:
a. preparing a water-in-oil (w/o) emulsion in which said active ingredient is present in either the oil or the water phase;
b. spraying said w/o emulsion in the presence of a carrier material comprising at least a water soluble matrix material and at least one emulsifier, to form agglomerates; and
c. optionally dispersing said agglomerates in an aqueous liquid, such as water or an aqueous solution.

14. Use of a method according to claim 4 for the encapsulation of an active ingredient.
